(19) United States
(12) Patent Application Publication
(10) Pub. No.: US 2021/0085702 A1
(43) Pub. Date: Mar. 25, 2021

(54) Use of Oligosaccharide Compounds for Treating Wounds of Arteriopathic Diabetic Patients

(71) Applicant: Urgo Recherche Innovation et Developpement, Chenove (FR)

(72) Inventor: Serge Bohbot, Levallois-Perret (FR)

(73) Assignee: Urgot Recherche Innovation et Developpement, Chenove (FR)

(21) Appl. No.: 16/613,474

(22) PCT Filed: May 16, 2018
(86) PCT No.: PCT/EP2018/062811

§ 371 Date: Nov. 14, 2019

(30) Foreign Application Priority Data
May 17, 2017 (FR) 1754363
Jun. 20, 2017 (FR) 1755620

(51) Int. Cl. A61K 31/7016; A61K 47/52; A61K 9/70; A61P 17/02

(52) U.S. Cl. CPC A61K 31/7016; A61P 17/02; A61K 9/703; A61K 47/52

(57) ABSTRACT

The subject of the present invention is a synthetic polysulfated oligosaccharide having 1 to 4 monosaccharide units, salts thereof, and complexes thereof, for use thereof for treating diabetic foot ulcer in arteriopathic patients.

The subject of the present invention is a synthetic polysulphated oligosaccharide having 1 to 4 monosaccharide units, salts thereof, or complexes thereof, for use thereof for treating diabetic foot ulcer in arteriopathic patients, in particular for activating the healing of diabetic foot ulcer in arteriopathic patients.

The healing of a wound is a natural biological phenomenon, mammalian tissues being capable of repairing localised lesions by means of repair and regeneration processes that are specific thereto.

The speed and quality of the healing of a wound are dependent on the general status of the organism affected, the aetiology of the wound, the condition and location of the wound, and the onset of infection or not, as well as genetic factors predisposing or not to wound healing disorders.

The natural healing of a wound takes place mainly according to three successive phases, each of these phases being characterised by specific cell activities which advance the repair process according to precise chronological sequences: the inflammatory phase, the granulation phase (or proliferative phase), and the maturation phase.

The first phase, the inflammatory phase, starts from the rupture of the blood vessels which triggers the formation of a clot (blood coagulation) primarily composed of fibrin and fibronectin, and which will form a provisional matrix. This matrix partially fills the lesion and will enable the migration within the damaged area of the inflammatory cells recruited to cleanse the wound. The platelets present will also release factors (e.g. cytokine, growth factors) enabling the recruitment of wound-healing cells such as inflammatory cells (neutrophils and macrophages), fibroblasts and endothelial cells.

The second phase corresponds to granulation tissue development. There is first observed a colonisation of the wound by fibroblast proliferation. Then, endothelial cell migration from healthy vessels will enable the formation of new blood cells (neovascularisation), or angiogenesis, of the damaged tissue. This angiogenesis step is fundamental for initiating wound healing. In the granulation tissue, the fibroblasts are activated and are differentiated into myofibroblasts exhibiting significant contractile properties, generated by actin microfilaments, enabling wound contraction.

The third phase of the repair process, maturation, is accompanied by a restructuring of the granulation tissue. A portion of the extracellular matrix is digested by proteases (essentially matrix metalloproteinases (MMPs) and elastases), and a progressive reorganisation of the extracellular matrix is observed. Progressively, type III collagen, the majority in granulation tissue, is replaced by type I collagen, the main matrix component of the dermis. At the end of the maturation phase, fibroblasts, myofibroblasts and vascular cells see the proliferation thereof and/or the activity thereof reduced. Then the excess cells die by apoptosis. In parallel with the restructuring of the extracellular matrix and with the apoptosis of the excess cells, the inflammatory state declines progressively. This is the longest phase: after about one year, the scar is restructured, it is no longer red, nor rigid, no longer causes pain and it flattens.

Nevertheless, certain types of wounds do not heal correctly, the 3 key phases of the process occurring abnormally, despite setting up the best possible physicochemical and biological conditions. Indeed, the speed and quality of the healing of a wound are dependent on intrinsic and extrinsic factors. This repair process may therefore be abnormally prolonged depending on:
the aetiology of the wound;
the condition therefore and the location thereof;
the onset of infection caused by the presence of certain infectious agents such as Staphylococcus aureus or Pseudomonas aeruginosa;
the existence of a pre-existing condition (such as diabetes, immune deficiency, venous insufficiency, etc.);
the external environment; or
genetic factors predisposing or not to wound healing disorders.

Among these wounds are found chronic wounds such as venous ulcers, pressure sores or characteristic wounds of diabetic subjects. Chronic wounds are defined by a lack of healing after an interval of 6 weeks from the appearance of the wound, regardless of the treatment applied. To treat this type of wounds, it may be crucial to accelerate the healing process.

Diabetic wounds are characterised as being a very specific type of chronic wounds, having their own specificities.

Diabetes is an increasingly widespread disease. According to recent estimations, about 285 million people worldwide suffer from it, a figure that should attain 439 million subjects by 2030. Diabetics are exposed to various complications associated with their disease. Among these include the increase in the incidence of cardiovascular accidents such as myocardial infarctions, strokes and microvascular complications such as retinopathy (potentially leading to blindness) and nephropathy (potentially leading to kidney failure). One of the most dramatic complications of diabetes is amputation. It is estimated that, worldwide, a person has a lower limb amputated due to diabetes every 30 seconds and that 85% of these amputations are preceded by a foot ulcer (International Diabetes Federation, IDF 2005). About 15% of those suffering from diabetes will develop a foot ulcer during their lifetime.

Diabetic foot ulcer (DFU) is defined as “full thickness wounds below the ankle in a diabetic patient irrespective of duration” (IDF, 2005). Indeed, the primary cause of the lack of healing of these diabetic wounds is associated with an exacerbated bioavailability of glucose. This induces numerous physiological and metabolic modifications, such as a thickening of the skin, significant oxidative stress potentially leading to neuropathy or arteriopathy. Arteriopathy and neuropathy are therefore two distinct risk factors of delayed diabetic wound healing, and more particularly diabetic foot wounds.

Diabetic foot ulcers are classified into different categories. There are, on one hand, diabetic foot ulcers of the neuropathic patient and, on the other, diabetic foot ulcers of arteriopathic patients which are clearly distinguished from diabetic foot ulcers of the neuropathic patient by the presence of ischaemic injury (characterised in particular by the decrease in the arterial blood supply to an organ). Thus, the neuropathic patient's diabetic foot is generally characterised by a warm, well-perfused foot and a palpable pedal pulse. Furthermore, the neuropathic patient exhibits a very pronounced, or even complete, loss of sensitivity at the level of their lesion. The ulceration is frequently located on the sole of the foot, under a neglected callus subject to strong plantar pressure. Conversely, the arteriopathic patient's diabetic foot is for its part cold with a pedal pulse that is not palpable. In general, it is painful, as the patient's sensitivity is in this case subject to little or no impairment. Nevertheless, this type of patient exhibits more or less pronounced impairment at the level of the wound vessels, ranging from a mere decrease in the blood supply to irreversible necrosis of the different vascular tissues potentially leading to amputation at the level of the impaired area or even beyond. For this type of patient, ulcers are situated in part at the level of the sole of the foot, but also frequently at the tips of the toes or in areas situated behind the heel. The major clinical differences between the forms of diabetic foot ulcers therefore require very distinct treatments. For the treatment of neuropathic ulcers, it is desirable to remove as much necrotic tissue and calluses as possible in order to restart the wound healing process. Conversely, an ischemic ulcer should not be debrided at risk of impairing the neoangiogenesis phase prior to the resumption of the conventional wound healing process. Furthermore, patients suffering from neuropathic ulcers heal on average two to four times more quickly than patients suffering from ischaemic ulcers, ischaemia significantly impeding the wound healing process and increased risks of infection, as it has emerged for example from the study “Comparison of characteristics and healing course of diabetic foot ulcers by etiological classification: neuropathic, ischemic and neuroischemic”, Totsu R R et al., J Diabetes Complications, 2014 July August; 28(4):528-35. Thus, the treatments proposed at the present time for diabetic foot ulcer in neuropathic patients are unsuitable and not transposable to arteriopathic patients. To date, for ischaemic wounds, no treatment (skin substitute, growth factor, dressing or medical device) has demonstrated the efficacy thereof.

Therefore, there remains to be sought an effective treatment of diabetic foot ulcer in arteriopathic patients in whom ischaemia complicates healing significantly.

The invention relates, according to a first aspect, to a synthetic polysulphated oligosaccharide having 1 to 4 monosaccharide units, salts thereof, or complexes thereof, for use thereof for treating diabetic foot ulcer in arteriopathic patients.

The invention also relates, according to a second aspect, to a pharmaceutical composition comprising a synthetic polysulphated oligosaccharide having 1 to 4 monosaccharide units, salts thereof, or complexes thereof, for use thereof for treating diabetic foot ulcer in arteriopathic patients.

Arteriopathic Patients

By “arteriopathic patients” is understood, according to the present application, diabetic patients presenting with ischaemia.

The distinction between neuropathic patients and arteriopathic patients presenting with ischaemia is clearly defined by the Texas classification system described in the publications “A comparison of two diabetic foot ulcer classification systems: the Wagner and the University of Texas wound classification systems”. Oyibo S O et al., Diabetes Care. 2001 January; 24(1): 84-8 and “A new classification of diabetic foot complications: a simple and effective teaching tool”, Dr Amit Kumar C Jain et al., The Journal of Diabetic Foot Complications, 2012; Volume 4, Issue 1, No. 1, Pages 1-5], which performs a two-dimension classification as recapitulated in the tables hereinafter:

WOUND DEPTH GRADE

| 0 | I | II | III |
|---|---|---|---|
| Pre or postulcerative | Superficial wound, | Wound penetration to | Wound penetrating to |
| lesion completely | not involving tendon, | tendon or capsule | bone or joint |
| epithelialised | capsule, or bone | | |

| STAGE | CLINICAL OBSERVATION |
|---|---|
| A | No infection or ischaemia |
| B | Infection but no ischaemia |
| C | Ischaemia but no infection |
| D | Infection and ischaemia |

Arteriopathic patients according to the present application are in categories IC and IIC whereas neuropathic patients are in class IA.

Ischaemia may be confirmed, firstly, by the ankle brachial pressure measurement less than or equal to 0.9, preferably less than 0.8 and/or the great toe brachial pressure index measurement less than or equal to 0.7.

Ischaemia may further be confirmed by an ankle systolic pressure greater than or equal to 70 mmHg and/or a great toe systolic pressure greater than or equal to 50 mmHg.

Measurement of one or more of these parameters makes it possible to identify the population of arteriopathic patients according to the invention.

According to a particular embodiment, arteriopathic patients according to the present application present with:
an ankle brachial pressure index greater than or equal to 0.9, preferably less than 0.8 or great toe brachial pressure index measurement less than or equal to 0.7, and
an ankle systolic pressure greater than or equal to 70 mmHg and/or a great toe systolic pressure greater than or equal to 50 mmHg.

According to a preferred embodiment, arteriopathic patients according to the present application present with:
either an ankle brachial pressure index ≤0.9, preferably less than 0.8, combined with a great toe systolic pressure ≥50 mmHg or, if the great toe measurement is not possible (amputation), with an ankle systolic pressure ≥70 mmHg;
or an ankle brachial pressure index >0.9, combined with a great toe systolic pressure ≥50 mmHg and with a great toe brachial pressure index ≤0.7.

Diabetic Foot Ulcer

The present invention proposes a synthetic polysulphated oligosaccharide having 1 to 4 monosaccharide units, salts thereof, or complexes thereof, for use thereof for treating diabetic foot ulcer in arteriopathic patients.

As described above, diabetic foot ulcer (DFU) is defined as “full thickness wounds below the ankle in a diabetic patient irrespective of duration”.

According to a particular embodiment, the diabetic foot ulcer treated within the scope of the present application is less than 5 cm² in size, i.e. the wound fits within a circle wherein the area is 5 cm².

According to a further preferred embodiment, the diabetic foot ulcer treated within the scope of the present application is not recalcitrant, i.e. it formed less than 6 months previously.

Synthetic Polysulphated Oligosaccharides Having 1 to 4 Monosaccharide Units

The oligosaccharides used within the scope of the present invention are synthetic oligomers formed from 1 to 4 monosaccharide units, preferably from 1 to 3 monosaccharide units, and more preferentially from 1 or 2 monosaccharide units, generally bonded to one another by an alpha or beta glycosidic bond. In other words, it consists of mono, di, tri or tetrasaccharides, and preferably of mono or disaccharides.

There is no particular limitation concerning the nature of the monosaccharide units of these polysaccharides. Preferably, they will consist of pentoses or hexoses. By way of example of monosaccharide, mention may be made of glucose, galactose or mannose. By way of example of disaccharide, mention may be made of maltose, lactose, sucrose or trehalose. By way of example of trisaccharide, mention may be made of melezitose. By way of example of tetrasaccharide, mention may be made of stachyose.

Preferably, the oligosaccharide is a disaccharide, and more preferably sucrose.

By “polysulphated oligosaccharide” is understood according to the present application an oligosaccharide wherein at least two, and preferably all the hydroxyl groups of each monosaccharide have been substituted by a sulphate group.

Preferably, the polysulphated oligosaccharide used within the scope of the present application is sucrose octasulfate.

The polysulphated oligosaccharides used within the scope of the present invention may be presented in salt or complex form.

By way of example of salts, mention may be made of alkaline metal salts such as sodium, calcium or potassium salts; silver salts; or indeed amino acid salts.

By way of example of complexes, mention may be made of hydroxyaluminium complexes.

Within the scope of the present invention, particularly preferred compounds are as follows:
potassium sucrose octasulfate salt;
silver sucrose octasulfate salt; and
hydroxyaluminium sucrose octasulfate complex, commonly known as sucralfate.

In particular, within the scope of the present invention, the polysulphated oligosaccharides used are preferably potassium salts rather than aluminium salts of sucrose octasulfate.

The polysulphated oligosaccharides used within the scope of the present invention may be presented in micronised powder form or in solubilised form.

An example of polysulphated oligosaccharide used within the scope of the present invention is potassium sucrose octasulfate salt (known as the abbreviation KSOS), marketed in the product Urgotul® Start by Laboratoires URGO.

According to a particular embodiment, the synthetic polysulphated oligosaccharide according to the invention is used at a concentration greater than or equal to 70 mg/mL, preferably greater than or equal to 100 mg/mL. According to a preferred embodiment, the synthetic polysulphated oligosaccharide according to the invention is used at a concentration between 100 mg/mL and 1000 mg/mL.

Composition

The invention also relates to a pharmaceutical composition comprising the synthetic polysulphated oligosaccharide described above, for use thereof for treating diabetic foot ulcer in arteriopathic patients.

Additional Active Substance

As a general rule, the oligosaccharide compounds according to the invention may be used alone or in a mixture of two or more thereof, or indeed combined with one (or a plurality of) further active substance(s).

As a general rule, the active substances are chosen from antibacterials, antiseptics, analgesics, anti-inflammatories, wound healing-promoting active substances, depigmenting agents, antipruritic agents, UV filters, soothing agents, moisturising agents, antioxidant agents, and mixtures thereof.

As a general rule, the active substances are chosen from:
antibacterials such as Polymyxin B, penicillins (Amoxycillin), clavulanic acid, tetracyclines, Minocycline, chlorotetracycline, aminoglycosides, Amikacin, Gentamicin, Neomycin, silvers and salts thereof (Silver sulfadiazine), probiotics, silver salts;
antiseptics such as thiomersal, eosin, chlorhexidine, phenylmercuric borate, hydrogen peroxide, Dakin's solution, triclosan, biguanide, hexamidine, thymol, Lugol, Povidone-iodine, Merbromin, Benzalkonium and Benzethonium Chloride, ethanol, isopropanol;
analgesics such as Paracetamol, Codeine, Dextropropoxyphene, Tramadol, Morphine and derivatives thereof, Corticosteroids and derivatives;
anti-inflammatories such as Glucocorticoids, non-steroidal anti-inflammatory drugs, Aspirin, Ibuprofen, Ketoprofen, Flurbiprofen, Diclofenac, Aceclofenac, Ketorolac, Meloxicam, Piroxicam, Tenoxicam, Naproxen, Indomethacin, Naproxcinod, Nimesulide, Celecoxib, Etoricoxib, Parecoxib, Rofecoxib, Valdecoxib, Phenylbutazone, niflumic acid, mefenamic acid;
wound healing-promoting active substances such as Retinol, Vitamin A, Vitamin E, N-acetyl-hydroxyproline, Centella Asiatica extracts, papain, silicones, essential oils of thyme, niaouli, rosemary and sage, hyaluronic acid, Allantoin, Hematite (Gattefossé), Vitamin C, TEGO Pep 4-17(Evonik), Toniskin (Silab), Collageneer (Expanscience), Timecode (Seppic), Gatuline skin repair (Gattefossé), Panthenol, PhytoCellTec Alp Rose (Mibelle Biochemistry), Erasyal (Libragen), Serilesine (Lipotec), Heterosides of Talapetraka (Beyer), Stoechiol (Codif), macarose (Sensient), Dermaveil (Ichimaru Pharcos), Phycosaccaride AI (Codif);
depigmenting agents such as kojic acid (Kojic Acid SL®-Quimasso (Sino Lion)), Arbutin (Olevatin®-Quimasso (Sino Lion)), the blend of sodium palmitoylpropyl and nymphaea alba extract (Sepicalm®-Seppic), undecylenoyl phenylalanine (Sepiwhite®-Seppic),
antipruritic agents: hydrocortisone, enoxolone, diphenyhydramine, topical H1 antagonist antihistamine
moisturising active substances such as xpermoist (Lipotec), Hyaluronic acid, Urea, fatty acids, Glycerine, Waxes, Exossine (Unipex)
UV filters such as Parsol MCX, Parsol 1789
soothing agents such as chamomile, bisabolol, xanthalene, glycyrrhebenic acid, tanactine (CPN), Calmiskin (Silab),
antioxidant agents, such as vitamin E.
   According to a preferred embodiment, the oligosaccharide compounds according to the invention may be used in combination with an antioxidant agent.

Galenic Formulation

The synthetic polysulphated oligosaccharides used within the scope of the present invention may be administered topically, and particularly in a galenic formulation, such as for example a gel, a solution, an emulsion, a cream, granules, capsules of variable sizes ranging from a nano or micrometre to a millimetre, which will enable the application thereof on the wound. Alternatively, the compounds used within the scope of the present invention may be used in a solution for subcutaneous injection.

If they are used in a mixture of two or a plurality thereof or indeed combined with one or a plurality of further active substances, these compounds may be incorporated in the same galenic formulation or in separate galenic formulations.

Obviously, the quantity of synthetic polysulphated oligosaccharides according to the invention used in the galenic formulation is adapted according to the kinetics sought as well as the specific constraints associated with the nature, solubility, heat resistance, etc. thereof.

Dressing

Preferentially, the synthetic polysulphated oligosaccharides used within the scope of the present invention, or a galenic formulation containing same, will be incorporated in a dressing.

The synthetic polysulphated oligosaccharides compounds, and particularly potassium sucrose octasulfate salt or a galenic formulation may be incorporated in any element of the structure of a dressing provided that this compound can come directly or indirectly into contact with the wound surface.

Preferably and in order to promote a rapid action, this compound (or a galenic formulation containing same) will be incorporated into the layer of the dressing that comes into contact with the wound or deposited on the surface of the dressing that comes into contact with the wound.

Advantageously, potassium sucrose octasulfate salt (or a galenic formulation containing same) may thus be deposited, continuously or discontinuously, on the surface intended to come into contact with the wound:
either in liquid form, for example by spraying a solution or suspension containing same;
or in solid form, for example by screening a powder containing same.

The layer or surface coming into contact with the wound may consist for example of an absorbent material such as a polyurethane hydrophilic absorbent foam; a textile material such as a compress, such as a non-woven material, a film, a fibre voile; an optionally absorbent adhesive material; an optionally adherent interface structure.

Alternatively, the layer or surface coming into contact with the wound may consist for example of a textile weft, preferably made of polyester as described in patent application WO 01/70285 or in patent application WO2013/093298 whereon will be covered, or coated, an elastomeric matrix comprising a synthetic polysulphated oligosaccharide having 1 to 4 monosaccharide units, salts thereof, or complexes thereof, in particular a potassium sucrose octasulfate salt, as described in patent application WO2008/149035 or in application WO 2014/009488.

The invention thus relates to a dressing comprising a textile weft coated with an elastomeric matrix comprising a synthetic polysulphated oligosaccharide having 1 to 4 monosaccharide units, salts thereof, or complexes thereof, in particular a potassium sucrose octasulfate salt, for use thereof for treating diabetic foot ulcer in arteriopathic patients.

As a general rule, the galenic formulation or the structure of the dressing may be adjusted to obtain a specific, quick or delayed, potassium sucrose octasulfate salt release profile, according to requirements.

Obviously, the quantity of potassium sucrose octasulfate salt used in the galenic formulation or in the dressing will be adapted according to the kinetics sought as well as specific constraints associated with the nature, solubility, heat resistance, etc. thereof.

By dressing is intended to denote, according to the present application, all types of dressings used for wound treatment.

Typically, a dressing comprises at least one optionally adhesive layer or matrix.

The synthetic polysulphated oligosaccharide compounds according to the invention, or a galenic formulation containing same, may be incorporated in any element of the structure of a dressing, for example in the matrix.

Preferably, and in order to promote a rapid action, this compound (or a galenic formulation containing same) may be incorporated in the layer of the dressing that comes into contact with the wound or deposited on the surface of the layer of the dressing that comes into contact with the wound.

Such deposition techniques are well-known to those skilled in the art and some are for example described in patent application WO 2006/007814.

According to an alternative embodiment of the invention, the synthetic polysulphated oligosaccharide compound according to the invention may be incorporated in a gelling fibre-based absorbent dressing, such as for example the product AQUACEL® marketed by CONVATEC.

Very often, when placing these dressings, the nursing staff hold the latter in position using a band or cover the latter with a secondary element such as a second absorbent dressing or a compression band. It is therefore useful for the dressing to remain fastened on the wound so that the nursing staff keep their hands free to position these secondary elements. As a general rule, any type of adhesive may be used for this purpose.

So as not to damage the healthy tissue or the wound margins, particularly when removing the dressing, an adhesive having the property of adhering to the skin without adhering to the wound will be preferred.

By way of example of such an adhesive, mention may thus be made of silicon or polyurethane elastomer-based adhesives, such as silicone or polyurethane gels, and hydrocolloidal adhesives.

Such hydrocolloidal adhesives particularly consist of an elastomeric matrix based on one or a plurality of elastomers chosen from poly(styrene-olefin-styrene) sequenced polymers in association with one or a plurality of compounds chosen from plasticisers, such as mineral oils, tackifying resins and, if required, antioxidants, wherein is incorporated a quantity, preferably small, of hydrocolloids (from 3 to 20% by weight) such as for example sodium carboxymethylcellulose or superabsorbent polymers such as the products such as the products marketed under the trade name LUQUASORB® by BASF.

According to a preferred embodiment, the synthetic polysulphated oligosaccharide compounds used within the scope of the present invention, or a galenic formulation containing same, will be integrated in a dressing comprising a hydrocolloidal adhesive, said polysulphated oligosaccharide being incorporated in said adhesive preferably at a quantity between 1 and 15% by weight, more preferably between 5 and 10% by weight, with respect to the weight of the adhesive.

The formulation of such hydrocolloidal adhesives is well-known to those skilled in the art and described for example in patent applications FR 2 783 412, FR 2 392 076 and FR 2 495 473.

The use of adhesive netting on the nonwoven makes it possible particularly advantageously to reduce or prevent the risk of tiny fibres of textile material coming into contact with the wound and sticking to tissue, thus triggering a painful sensation on removal, or even impeding the wound healing process.

According to a preferred alternative embodiment of the present invention, the synthetic polysulphated oligosaccharide compound according to the invention is incorporated in such an adhesive at a concentration compatible with the solubility thereof and the heat resistance thereof.

Based on these criteria, the synthetic polysulphated oligosaccharide compound according to the invention is used preferably at a quantity between 1 and 15% by weight, and more preferably between 5 and 10% by weight, with respect to the total weight of the adhesive.

If it is sought to increase the absorption of this nonwoven dressing, there may be associated therewith an additional absorbent layer, and preferably a non-gelling absorbent layer, such as in particular a compress such as that used in the product URGOTUL® Duo or URGOTUL® Trio, an absorbent hydrophilic foam, preferably a hydrophilic polyurethane foam having an adsorption capacity greater than that of the nonwoven such as that used in the product CELLOSORB®.

According to a preferred embodiment, the synthetic polysulphated oligosaccharide compound according to the invention is incorporated in a nonwoven dressing, associated with an additional absorbent layer, and preferably a non-gelling absorbent layer, such as in particular a compress.

According to a further preferred embodiment, the synthetic polysulphated oligosaccharide compound according to the invention is incorporated in a nonwoven dressing, associated with an additional absorbent layer, and preferably a non-gelling absorbent layer, such as in particular an absorbent hydrophilic foam, preferably a hydrophilic polyurethane foam having a greater absorption capacity than that of the nonwoven.

The nonwoven and the foam may be associated by techniques well-known to those skilled in the art, for example by hot rolling using a hotmelt powder based on TPU/polycaprolactone polymers.

This technique is routinely used for binding nonwovens intended for the medical market to one another.

Finally, this foam and the nonwoven (when the latter is used alone) may be covered with a substrate to protect the wound from the outside.

This substrate may be greater in size than that of the other layers and rendered adhesive continuously or discontinuously on the face thereof coming into contact with the wound so as to optimise the securing of the dressing during the use thereof, in particular if the wound is situated on non-planar body areas.

This substrate and the adhesive thereof are preferably impermeable to fluids but very permeable to water vapour so as to enable optimal management of the exudates absorbed by the dressing and prevent maceration problems.

Such substrates are well-known to those skilled in the art and consist for example of breathable and impermeable films such as polyurethane films, foam/film or nonwoven/film complexes.

Additives

Besides the active agents, the oligosaccharide compounds according to the invention may be used combined with one (or a plurality) of additives routinely used in the preparation of dressings. These additives may particularly be chosen from fragrances, preservatives, vitamins, glycerine, citric acid, etc.

The activity of the synthetic polysulphated oligosaccharides according to the invention was demonstrated in the following non-limiting examples.

### EXAMPLE: DEMONSTRATION OF THE EFFECT OF POTASSIUM SUCROSE OCTASULFATE SALT (KSOS) for Treating Diabetic Foot Ulcer in Arteriopathic Patients

A double-blind randomised controlled trial was conducted based on 240 arteriopathic diabetic patients divided into two parallel groups: a control group of 114 patients and a treated group of 126 patients. The groups are also divided into subgroups according to the size of the wound and the recalcitrant nature thereof. Thus, patients whose ulcers have a size of less than 5 cm², i.e. the wound fits within a circle wherein the area is 5 cm² are distinguished from those having a size between 5 and 30 cm². A distinction is also made between patients whose ulcer treated within the scope of the present application is not recalcitrant, i.e. it formed less than 6 months previously, and patients whose ulcer is recalcitrant, i.e. it formed more than 6 months previously.

Arteriopathic patients are patients presenting with type 1 or 2 diabetes and glycated haemoglobin (HbA1c)≤10%, and presenting with a diabetic foot ulcer of a surface area between 1 and 30 cm².

The patients are in categories IC and IIC of the Texas classification and present with ischaemia characterised:
Either by an ankle brachial pressure index ≤0.9 combined with a great toe systolic pressure ≥50 mmHg or, if the great toe measurement is not possible (amputation), with an ankle systolic pressure ≥70 mmHg;
Or by an ankle brachial pressure index >0.9, combined with a great toe systolic pressure ≥50 mmHg and with a great toe brachial pressure index ≤0.7.

After cleaning the wound with a saline solution, there was applied to the patient, either a dressing comprising a potassium sucrose octasulfate salt or a dressing having the same structure but not containing polysulphated oligosaccharide.

The dressing consists of a polyester openwork textile weft coated with an elastomeric matrix; this matrix is particularly described in patent application WO 201009488.

The dressing is changed every two to four days according to the degree of exudation of the wound and the patients are treated for not more than 20 weeks.

Every month, a clinical wound healing assessment is conducted based on the following criteria:
treatment tolerance
condition of perilesional skin
pain of wound
clinical examination of wound (necrotic tissue, granulation tissue, pressure sores, etc.)
wound characterisation (wound size, general appearance, analysis of margins, perilesional skin, outline of wound perimeters to track changes over time)

Results:

According to the present example, by the term “wound closure” is understood any healed wound, i.e. up to complete epithelialisation of the dermis, the latter being confirmed two weeks after the 20^{th} week by specialised doctors.

As illustrated in FIG. 1, the present study demonstrated that wounds treated with the dressing according to the invention comprising a potassium sucrose octasulfate salt exhibit significantly enhanced ulcer healing, characterised particularly by a wound closure percentage of 47.6% versus merely 29.8% in the control group treated with the comparative dressing having the same structure but not containing polysulphated oligosaccharide.

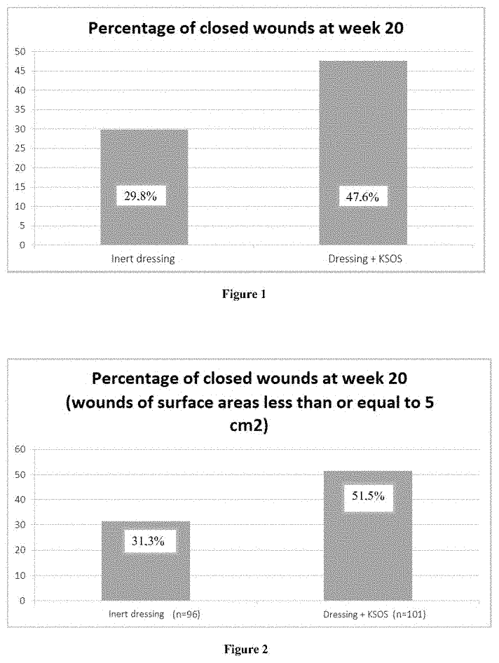

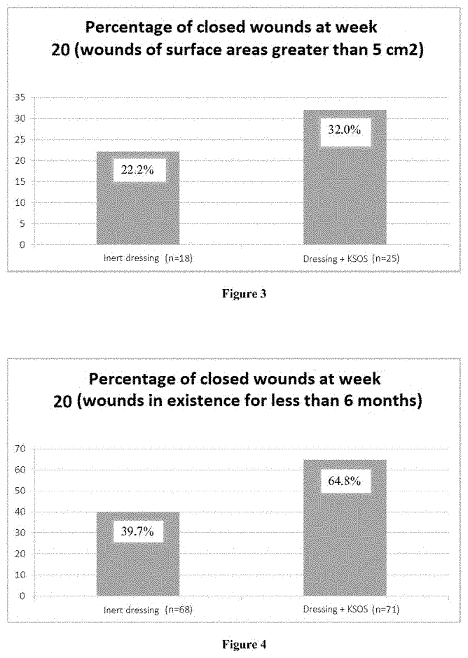

It is further clear from FIGS. 2 and 3 that when the ulcers treated with the dressing according to the invention comprising a potassium sucrose octasulfate salt have a size less than 5 cm², i.e. the wound fits inside a circle wherein the area is 5 cm², they exhibit significantly enhanced healing, characterised particularly by a wound closure percentage of 51.5% versus 31.3% in the control group.

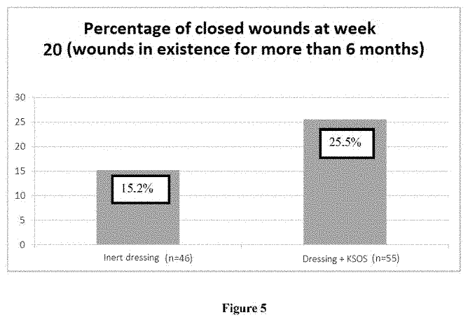

FIGS. 4 and 5 also show that when the ulcers treated with the dressing according to the invention comprising a potassium sucrose octasulfate salt are not recalcitrant, i.e. they formed less than 6 months previously, they exhibit significantly enhanced healing, characterised particularly by a wound closure percentage of 64.8% versus 39.7% in the control group.

TABLE 1
hereinafter cross-references the data from FIGS. 2 to 5:

| | | | Difference between |
|---|---|---|---|
| | | | dressing + KSOS |
| | Inert dressing | Dressing + KSOS | group and control |
| | control group | group | group |
| Total | 29.8% (34/114) | 47.6% (60/126) | 17.8 |
| Wounds of surface areas ≤ | 42.1% (24/57) | 69.5% (41/59) | 27.4 |
| 5 cm² and in existence | | | |
| for less than 6 months | | | |
| (n = 116) | | | |
| Wounds of surface areas > | 27.3% (3/11) | 41.7% (5/12) | 14.4 |
| 5 cm² and in existence | | | |
| for less than 6 months | | | |
| (n = 23) | | | |
| Wounds of surface areas ≤ | 15.4% (6/39) | 26.2% (11/42) | 10.8 |
| 5 cm² and in existence | | | |
| for more than 6 months | | | |
| (n = 81) | | | |
| Wounds of surface areas > | 14.3% (1/7) | 23.1% (3/13) | 8.8 |
| 5 cm² and in existence | | | |
| for more than 6 months | | | |
| (n = 20) | | | |

It is clear from this table that patients presenting with a non-recalcitrant ulcer (formed less than 6 months previously) of less than 5 cm² in size exhibit particularly enhanced healing when they are treated with the dressing according to the invention comprising a potassium sucrose octasulfate salt.

It is clear from table 2 below that patients treated with the dressing according to the invention exhibit a significant reduction in their treated wound surface area (expressed as a % reduction or in cm² of surface area reduction), said treated wound surface area indeed exhibiting a median reduction of 1.76 cm² for patients having the dressing according to the invention applied, versus a median reduction of 1.23 cm² for patients on whom the control dressing is applied.

TABLE 2

| | Inert dressing control group | Dressing + KSOS group |
|---|---|---|
| | (n = 114) | (n = 126) |
| Reduction in wound surface | −42.3 +/− 114.8 | −71.7 +/− 46.5 |
| area from D0 to end of week | | |
| 20 (% of surface area) | | |
| Median value | −89.9 | −98.1 |
| Limit values | −100 to 707.8 | −100 to 222.1 |
| Reduction in wound surface | −2.28 +/− 5.51 | −3.17 +/− 5.22 |
| area from D0 to end of week | | |
| 20 (cm² of surface area) | | |
| Median value | −1.23 | −1.76 |
| Limit values | −28.80 to 14.44 | −32.12 to 15.53 |

Finally, it is clear from table 3 below that the mean wound closure interval between patients treated with the dressing according to the invention is significantly quicker (119.7+/−4.7 days on average) than that of the group of patients treated with the control dressing (180.5+/−8.7 days on average):

TABLE 3

| | | | Difference between |
|---|---|---|---|
| | | | Dressing + KSOS |
| | | | group (n = 126) and |
| | Inert dressing control | Dressing + KSOS | inert dressing control |
| | group (n = 114) | group (n = 126) | group (n = 114) |
| Wound closure | 180.5 +/− 8.7 | 119.7 +/− 4.7 | 60.8 |
| interval (days) | | | |

Thus, the present study helped demonstrate that, regardless of the sex of the patients treated, whether they present with a recalcitrant ulcer or not, and regardless of the size of the ulcer treated, a significantly greater number of patients treated with the dressing according to the invention comprising a potassium sucrose octasulfate salt exhibit complete healing of the ulcer compared to patients treated with the comparative dressing having the same structure but not containing polysulphated oligosaccharide. Furthermore, the healing of wounds treated with the dressing according to the invention is quicker than that of wounds treated with the comparative dressing not containing polysulphated oligosaccharide, which is demonstrated by the wound closure percentage measurements made. All of the results of the present study will be the subject of a publication under the reference “Explorer study”.

As a result, polysulphated oligosaccharides, and in particular potassium sucrose octasulfate salt, enable rapid and effective treatment of diabetic foot ulcer in arteriopathic patients, in particular presenting with ischaemia.

1. A method of treating a diabetic foot ulcer in an arteriopathic patient comprising administering a synthetic polysulphated oligosaccharide having 1 to 4 monosaccharide units, salts thereof, or complexes thereof to the arteriopathic patient.

2. The method according to claim 1, wherein the synthetic polysulphated oligosaccharide is administered in a concentration sufficient to promote healing of the diabetic foot ulcer.

3. The method according to claim 2, wherein the concentration of the polysulphated oligosaccharide is greater than or equal to 70 mg/mL.

4. The method according to claim 1, wherein the polysulphated oligosaccharide comprises 1 to 3 monosaccharide units, 1 or 2 monosaccharide units selected from pentoses and hexoses, salts and complexes of these compounds.

5. The method according to claim 1, wherein the polysulphated oligosaccharide is selected from:
potassium sucrose octasulfate salt;
silver sucrose octasulfate salt; and
hydroxyaluminium sucrose octasulfate complex.

6. The method according to claim 1, wherein the polysulphated oligosaccharide is potassium sucrose octasulfate salt.

7. The method according to claim 1, wherein the polysulphated oligosaccharide is administered in the form of a composition selected from a gel, a solution, an emulsion, a cream, granules or capsules enabling application directly on the wound.

8. The method according to claim 7, wherein composition is a pharmaceutical composition comprising a synthetic polysulphated oligosaccharide having 1 to 4 monosaccharide units, salts thereof, or complexes thereof.

9. A method according to claim 1, wherein the synthetic polysulphated oligosaccharide having 1 to 4 monosaccharide units, salts thereof, or complexes thereof is potassium sucrose octasulfate salt present in a dressing.

10. The method according to claim 9, wherein the dressing is a textile weft coated with an elastomeric matrix.
